(19) United States
(12) Patent Application Publication
(10) Pub. No.: US 2018/0303330 A1
(43) Pub. Date: Oct. 25, 2018

(54) Image-recording device, Balloon for operation with an image-recording device, method for operating an image-recording device, and control program for an image-recording device

(71) Applicant: Ivoclar Vivadent AG, Schaan (LI)

(72) Inventors: Gottfried Rohner, Altstätten (CH); Ronny Watzke, Feldkirch (AT); Theresa Senti, Schaanwald (LI)

(21) Appl. No.: 15/751,271

(22) PCT Filed: Aug. 9, 2016
(86) PCT No.: PCT/EP2016/068971

§ 371 Date: Jun. 5, 2018

(30) Foreign Application Priority Data
Aug. 11, 2015 (EP) 15180555.3

(51) Int. Cl. A61B 1/24; H04N 13/221; G06T 7/521; G06T 7/73; G06T 7/00

(52) U.S. Cl. CPC A61B 1/24; H04N 13/221; G06T 7/521; G06T 2207/30004; G06T 7/0012; G06T 2207/10012; G06T 7/74

(57) ABSTRACT

The invention relates to an image-recording device (10), comprising a scanning-head guide (18) for moving the scanning head (20) across a scanning region (42) of a cavity, which scanning region (42) extends around the scanning head (20). A planar or film-like, elastically extensible material extends between the scanning head (20) and the scanning region (42), which material can be pressed against the scanning region (42) by the application of overpressure in the manner of a balloon (12). The scanning-head guide (18) extends through the balloon connection (16) of the balloon (12) to the scanning head (20). In particular, a control device (32) determines the shape of the cavity (40) against which the material rests from the deformation of the material while the overpressure is present.

The invention relates to an image-recording device according to the preamble of Claim 1, a balloon according to the preamble of Claim 15, a method for operating an image-recording device according to the preamble of Claim 16 or 19, respectively, and a control program for an image-recording device according to the preamble of Claim 2229.

For the analysis of measuring signals, it is known to place inflatable balloons around recording coils. Inflating is done via the balloon connection, the connections for the sensor also being passed therethrough.

Such a solution may for example be taken from DE 42 33 809 A1 for the medical field. The solution allows protection of the sensitive sensor, which, for example, may be a NMR recording device.

In some cases, it is desirable to acquire the internal structure of a partially jagged cavity. For this, typically stereometric methods with scanning heads are employed, which are spaced apart from each other by a predetermined extent. The surface to be recorded of the cavity, the so called scanning region, is illuminated by an illumination device at the scanning head, and it is intended to thus acquire the cavity.

However, it often occurs, that the scan result is not satisfying, despite the use of elaborate scanners, so that attempts have been made to further improve the scanning head with appropriately high cost. However, the attempts made so far gave little satisfying results, especially in three-dimensional elaborately shaped cavities.

In order to nevertheless allow fairly precise surface scanning of the cavity, i.e. to receive the scan range, it has been suggested to make use of different frequency ranges of the electromagnetic radiation. For example, for a humid surface, it may be beneficial to use visible light or UV light. Meanwhile, the reflection characteristics of certain materials, of which the cavity may consist, are, on the one hand, better with ultra sound, but, on the other hand, are as well better with X-rays.

In this context, it of disadvantage that scanners having different frequency ranges are required to be provided, making the solution in total more expensive and moreover, making it in part not feasible any more.

It thus an object of the invention, to provide an image-recording device according to the preamble of Claim 1, a balloon for operating with an image-recording device according to the preamble of Claim 15, a method for operating an image-recording device according to the preamble of Claim 16 or 19, respectively, and a control program for an image-recording device according to the preamble of Claim 229, which are significantly improved in scan result without considerable capital expenditure.

According to the invention, this object will be solved by Claim 1, 15, 16, 19, or 22, respectively. Advantageous embodiments will arise from the SubClaims.

According to the invention, it is especially favorable for the film-like or planar, elastically extensible material to tightly rest against the scanning region. This is assured by the overpressure, which in addition preferably is adjustable. By comparing the scanning result with a lower overpressure and the scanning result with a higher overpressure it is possible to determine in as much the scanning region is deformable, i.e. in as much it consists of a hard substance or a soft substance.

If the scanning region or the cavity adjacent to the scanning region, respectively, comprises gaseous inclusions, the substance will be pressed outwards by the overpressure in the balloon. If, on the other hand, a jelly-like deformable substance is present, the surface is equalized by the pressure, so that it is less jagged that with the slight overpressure.

According to the invention, it is provided for the scanning region that almost extends in the type of a jagged, but essentially spherical cavity around the scanning head, to be completely covered by the extensible material that is part of a balloon.

The scanning head with the scanner(s) is inserted through the elastically extensible balloon connection, and subsequently to this, the balloon connection is sealed against the scanning head guide that extends therethrough.

The balloon now is pressurized through the scanning head guide. In this way, it rests against the scanning region.

On the balloon film that preferably forms the elastically extensible material, a reference pattern is applied. The configuration thereof in the depressurized state of the balloon has been known. The reference pattern deforms due to extension of the balloon, so that the individual reference lines and reference points will move away from each other. From this extension, which each occurs punctual, the shape of the cavity in detail may only be indirectly derived, without the requirement of failing stereoscopic examination.

The reference pattern may be applied onto the balloon in any suitable manner. Upon application from inside it is beneficial for the balloon film to be thin, for example 80 μm, to maintain exactness of measurement. If applied from outside, complete transparency of the film is beneficial, for example with a transmittance of 0.9 or an even higher transmittance. Here also, a thin balloon film is beneficial.

The control device of the image-recording device according to the invention acquires the extension of the reference pattern at every point of the scan range that was recorded by the scanner(s). From the extension, the three-dimensional shape of the scanning region will now be calculated, wherein it is beneficial to receiving the extension that it is two-dimensional and may also be two-dimensionally received. In this respect, it is important to the reference pattern to represent a real pattern, i.e. not only adjacently extending lines without intersection.

In selecting the pattern, it is beneficial to optimize software-related detectability. For example, a geometrically uniform pattern with dot-dashed lines in one direction and dashed lines in the transverse direction may be beneficial for this. But any other regular, but also irregular reference patterns are also feasible. An example is a hexagonal pattern a with a leg length of 200 μm.

The line width of the reference pattern may, in wide areas, be adopted to the resolution of the scanners of the scanning head. Thus, for example, a very fine line width of 15 μm may enable very precise reception.

In an advantageous configuration, it is provided for the material to be formed as a flexible plastic film, which especially is water and/or grease resistant and preferably consists of elastomer, silicone or latex. The elastically extensible material may be formed as a plastic film and may be formed as a disposable part.

The scanning region may also be directly provided with a reference pattern, e.g. by optically reflecting such a pattern onto the scanning region.

Preferably, the balloon film is transparently formed and especially has a strength of less than 300 μm, preferably about 50 μm.

In another advantageous configuration it is provided for an overpressure source to be provided, yielding an essentially constant pressure and being coupled to the balloon connection, wherein especially a pressure relief valve is in flow connection with the balloon connection or the overpressure source.

In another advantageous configuration, it is provided for the balloon film to be collapsible, for example by application of vacuum, and is removable from the cavity through the balloon connection.

In another advantageous configuration, it is provided that the balloon film, upon applying overpressure outside the cavity, occupies a spherical shape with a protruding dome-shaped external shape to the spherical shape in the direction towards the balloon connection.

In another configuration of the invention, a method for operating an image-recording device is provided, comprising a scanning head and a scanning head guide with a scanning aid transparent to scanning radiation, wherein the method is characterized in that the scanning aid is introduced into a cavity to be scanned in a soft or liquid state, that the cavity is reduced upon curing the scanning aid or while curing the scanning aid, that following curing, the scanning aid is removed, that subsequently the scanning head is introduced into the scanning aid and the surface of the scanning aid will be scanned.

According to the invention, it is essential that during the scanning operation the position of the scanning head in relation the cavity will not be changed. This, for example, may be realized by a reference object that is disposed in the balloon and adjacent to a sub-region of the cavity. When moving the scanning head, also movement relative to the reference object occurs, which in turn may be eliminated by calculation.

Such a reference object, of which several may also be arranged dispersed above the cavity, may also serve for improved alignment of the collected scanning images, the so called stitching. The reference object may occasionally also be attached inside the film.

According to the invention, stereometric acquisition of the scanning region or indirectly, by acquisition of the reference pattern adjacent thereto, respectively, is not required. For the sake of improving exactness, it may be beneficial to trigonometrically acquire at least one point of the reference pattern by two spaced apart scanners.

The elastically extensible material may be part of a balloon onto which the reference pattern is printed. The pressure may be realized at low cost, especially if attachment on the outside of the balloon film is realized. In this case, the balloon may also be formed as a disposable part, which quickly may be put over the scanning head.

In another advantageous configuration, it is provided for the balloon to initially be put under a limited overpressure, which may also be adjustable, for example via a pressure relief valve. The real extension of the reference pattern that is applied on the balloon film is then done by selective volume reduction of the cavity, for example, if it consists of two opposite half-shells.

In another advantageous configuration, it is provided, for the image-recording device to record the volume increase of the balloon and/or the extension of the balloon film during inflating. From this extension, the strength of the balloon film after inflating may be deducted at the considered position, as the extension, in a first approximation, is inversely proportional to the wall strength.

For this, the control device determines the enlargement of the he reference pattern upon inflating. The balloon film will be deformed in relation to an approximate spherical shape as soon as it becomes rested against the scanning region. Up to this point, an essentially uniform extension of the balloon film and an appropriate two-dimensional enlargement of the reference pattern occurs, which enlargement is acquired by the image-recording device.

Simultaneously, the balloon film becomes thinner, so that with a known initial wall strength of e.g. 150 μm and a—linear—reference pattern enlargement by the factor of 3, the remaining balloon film wall strength may be calculated to be 50 μm.

As soon as the balloon film, in one position, becomes to rest against the scanning region, the reference pattern will not significantly be further enlarged at that position. According to the invention, this will be acquired.

For acquisition of extension in the space, a polar coordinate system is suitably taken as a basis.

If the pressure in the balloon is further increased, and, for example, if the balloon at all sides rests on the scanning region little further enlargement of the reference pattern occurs that corresponds to the resilience of the scanning region at that position.

From the ratio of pressure increase—corresponding to the force applied to the position of the scanning region—and extension in the tangential direction—corresponding to the path in the radial direction—deformation of the scanning region at any point by the applied force—and thus, as far applicability of Hook's Law may be presumed, the local modulus of elasticity may be determined.

Moreover, reduction of the wall strength of the balloon film—as far as required—may be included into the calculation. The wall strength especially is of importance if the reference pattern is applied on the internal surface of the balloon, as the scanning region then is spaced apart from the reference pattern in the radial direction around the wall strength.

Upon printing onto the external surface of the balloon, this also will be tangentially expanded, thus becoming thinner; this thickness reduction may also be included in the calculation, if required.

According to the invention, a scanning device with a resolution and exactness between 10 and 50 gm is preferred. The printed reference pattern may have a strength of 30 μm and undergoes thickness reduction to 10 μm, while tangential-linear balloon extension is by the factor of 3. The image-recording device senses the radial internal surface of the print, so that der scanning region is radially by 10 μn further to the exterior than it is given by the acquisition by the die image-recording device. Thus, this difference only is of weight in image-recording devices having very high exactness, and may typically be neglected.

Further advantage, details and characteristics will arise from the following description of several illustrated embodiments of the invention by way of the drawings, wherein:

FIG. 1 shows a schematic view of an embodiment of an image-recording device according to the invention;

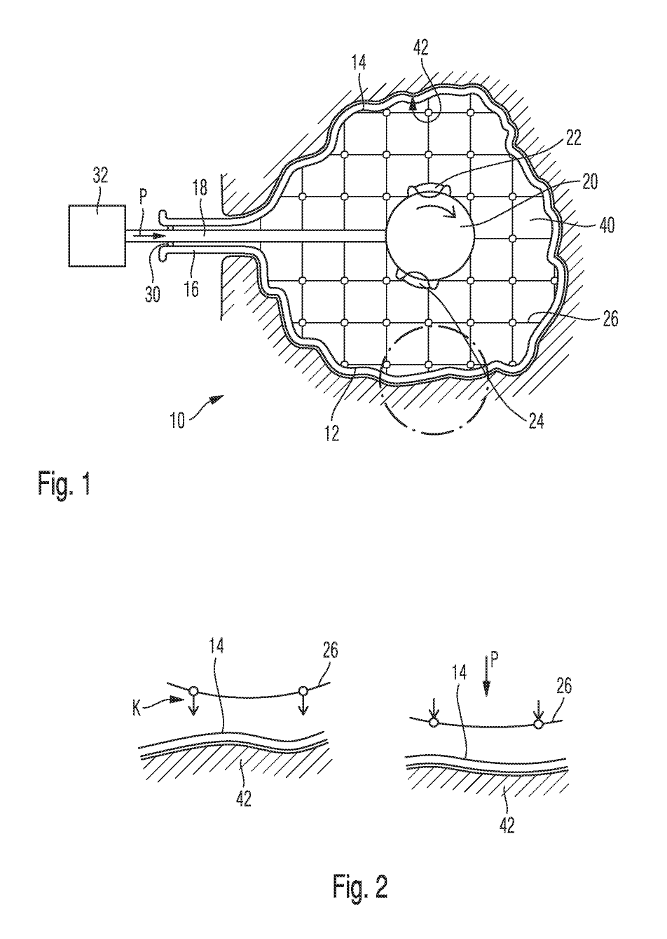

FIG. 2 shows a detail of FIG. 1 in two states; and

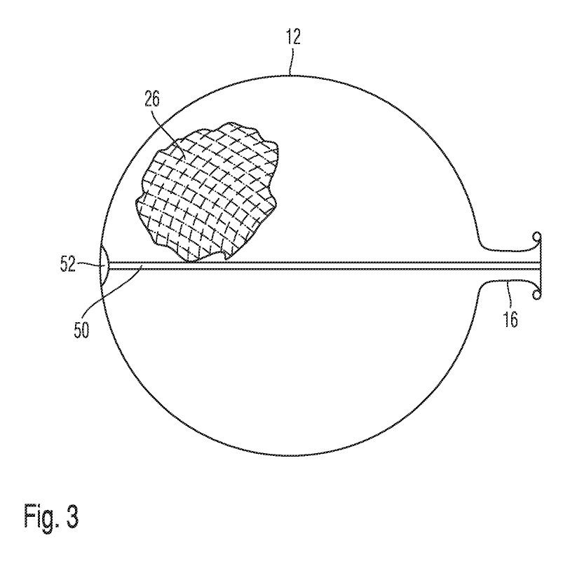

FIG. 3 shows another embodiment of a detail of the image-recording device according to the invention, i.e. of the balloon having the reference pattern and a pulling device.

From FIG. 1, an image-recording device 10 according to the invention may be seen in schematic representation. It comprises a balloon 12, which consists of a balloon film 14, which forms the elastically extensible material. The balloon 12 has a balloon connection 16 known per se. A scanning head guide 18 passes therethrough securely holding and guiding a scanning head 20 in the interior of the balloon.

Multiple scanners are attached to the scanning head 20 whereof two scanners 22 and 24 are represented herein. A plurality of scanners may in fact be provided, for example 100, whereas, with a smaller number of scanners, said scanners preferably are movably supported at the scanning head 20.

The scanners are to acquire the entire internal space of the balloon film 14. The balloon film 14 comprises a reference pattern 26 that, in the illustrated embodiment, is formed according to the type of a meshwork extending in uniform line spacing across the balloon film.

A seal 30 is provided surrounding the scanning head guide 18 sealing the balloon connection 16 against the ambient air. A control device 32 is provided exterior of the balloon. In any case, said control device analyses the images acquired by the scanners and, in the example embodiment represented, also controls an overpressure P, with which the balloon 12 is inflated.

By inflating, the balloon film 14 rests against a cavity 40 in a tight and contour-following manner. Thus, the reference pattern 26, in the deformed state, extends along a scanning region 42. According to the invention, the shape of the scanning region may be calculated in detail from the deformation of the reference pattern 26.

Moreover, the solution according to the invention is especially suitable for acquiring the deformability of the cavity. For this, reference is made to FIG. 2. The section of the balloon film 14 represented therein is pressed to the scanning region 42 with the force K, which depends on the magnitude of the overpressure P. In the state represented on the left-hand side in FIG. 42, the balloon film 14 rests against the contour of the scanning region 42 in contour-following manner, and appropriate deformation of the reference pattern 26 occurs.

When a higher pressure P is exerted, the elastic scanning region 42 is displaced further downwards, and the reference pattern 26 extends in a less strongly structurized manner, wherefrom the elastic deformability may be deducted.

From FIG. 3 it may be seen, in which way the reference pattern 26 may be attached to the balloon 12, in a modified configuration. Even though, for the sake of simplicity of the representation, the reference pattern is represented herein as being realized with a line spacing of for example 500 μm, it is understood that in practice, significantly smaller patters are possible, for example with a structure spacing of 50 μm and a line width von 15 μm.

As it may be seen from FIG. 3, a dot-dashed line configuration is selected in one dimension, and a dashed one is selected in the other dimension. From this, the relative orientation in space may be acquired via the scanning head 20.

In the illustrated embodiment represented according to FIG. 3, a pulling device 50 extends from the distant region 52 of the balloon opposite to the balloon connection 12 through the balloon connection 16 to selectively separate this region 52 from the cavity 40.

This solution, for example, is beneficial, if the cavity is the mouth of a patient and scan of the mouth's interior space is to be done, without causing a gag reflex to arise by the overpressure of the balloon.

In an advantageous configuration, the balloon 12 is inflated such that it completely rests against the interior of the cavity. By reducing volume of the cavity further pressure increase subsequently is done, which is acquirable by the scanning head 20.

The scanning head 20 may be operated in any suitable wavelength range. Herein, electromagnetic radiation such as light radiation is conceivable, for which an additional light source is preferred, illuminating the balloon's inner space and being attached to the scanning head 20. Use of X-rays or ultra sound is also possible instead or in addition thereto.

In another embodiment it is provided to initially allow introduction of a scanning aid, transparent to the scanning radiation, into the cavity and subsequently allow curing thereof.

The scanning aid comprises an introduction opening for a scanning head. It is cured in the cavity 40, thereby deforming the reference pattern applied to the surface of the scanning aid. Following at least partial curing it will be removed—eventually while elastically compressing or opening the cavity—and following this, the scanning head is introduced into the scanning aid and the surface of the scanning aid is scanned from the interior.

1. An image-recording device comprising
a scanning head (20),
a scanning head guide (18) for moving the scanning head across a scanning region of a cavity, which scanning region extends around the scanning head,
a balloon (12),
wherein the balloon comprises a planar or film-like, elastically extensible material that extends between the scanning head (20) and the scanning region (42), which elastically extensible material can be pressed against the scanning region (42) in a balloon (12)-type manner using overpressure, and
wherein the scanning head guide (18) passes through a balloon connection (16) of the balloon (12) to the scanning head (20), and wherein a control device (32) determines the shape of the cavity (40) against which the elastically extensible material rests from the deformation of the elastically extensible material in the presence of overpressure of the elastically extensible material.

2. The image-recording device according to claim 1,
wherein the elastically extensible material is provided with a reference pattern (26) that, in relation to the balloon (12), is applied on the interior or exterior side of the elastically extensible material.

3. The image-recording device according to claim 1,
wherein a flexible meshwork or a flexible grid frame is provided, which is applied to the scanning region (42) and
wherein the elastically extensible material is pressed to the scanning region (42), which serves as a reference pattern (26).

4. The image-recording device according to claim 1,
wherein the scanning region (42) is provided with a reference pattern (26).

5. The image-recording device according to claim 4,
wherein the control device (32) increases the overpressure until the reference pattern (26), during scanning, changes to not more than to a predetermined amount.

6. The image-recording device according to claim 1,
wherein the balloon film (14), at the side opposite to the balloon connection (16), comprises a pulling device (50), by which the film, at overpressure, is pullable towards the balloon connection (16) and/or is fixable at the balloon connection (16).

7. The image-recording device according to claim 1,
wherein the balloon film (14) is stiffened at a region opposite to the balloon connection (16) and is biased towards the balloon connection (16), so that, without overpressure, a dome arises, which persists even at the overpressure.

8. The image-recording device according to claim 1,
wherein the balloon film (14) comprises a wall decreasing in thickness starting from the balloon connection (16) and a wall strength, in connection thereto, increasing up to a position opposite to the balloon connection (16), wherein the maximum wall strength is less than 500 μm.

9. The image-recording device according to claim 4,
wherein the scanning head (20) comprises at least two scanners (22, 24) spaced apart from each other, which trigonometrically acquire the distance to at least one point of the reference pattern (26).

10. The image-recording device according to claim 1,
wherein the balloon film (14) is transparent to a radiation emitted from the scanning head (20) and/or to an irradiation reflected from the scanning region (42), with a transmittance of more than 0.8.

11. The image-recording device according to claim 4,
wherein the reference pattern (26) absorbs a radiation emitted from the scanning head (20) with an absorption coefficient of 100/mm and a reflectance of less than 5%.

12. The image-recording device according to claim 4,
wherein the reference pattern (26) comprises a strength of less than 50% of the strength of the film.

13. The image-recording device according to claim 4,
wherein the reference pattern (26) has an irregular geometric structure, having multiple prints differing among each other, which multiple prints extend in the space across a predominant part of the elastically extensible material.

14. The image-recording device according to claim 4,
wherein the reference pattern (26) has a plurality of specific reference points, which are oriented in advance towards prominent points of the cavity (40), by manually displacing the elastically extensible material towards the points.

15. A balloon and image-recording device combination comprising
an image recording device and
a balloon, wherein the balloon comprises a balloon connection, via which the balloon is overpressurized,
wherein a reference pattern (26), is applied to at least a portion of the balloon (12).

16. A method for operating an image-recording device, comprising
applying a balloon (12) comprising an extensible film to which a reference pattern (26) is applied, onto a scanning head (20) of an image-recording device (10) and
introducing the balloon and scanning head into deformable cavity (40) such that the balloon (12) is overpressurized via a balloon connection (16), so that the balloon rests against the cavity (40), and
wherein a scan is subsequently started and performed across a predetermined scanning region (42).

17. The method according to claim 16,
wherein a first scan is performed at a first overpressure, at which the balloon film (14) rests against the cavity (40), and a second scan is performed at a second overpressure, at which the balloon film (14) conforms to the cavity (40).

18. The method according to claim 16,
wherein a control device (32) of the image-recording device (10) determines the resilience of the surface and/or the change of the surface from the deformation of the reference pattern (26) corresponding to the deformation of the surface of the cavity (40).

19. A method for operating an image-recording device, with a scanning head and a scanning head guide, which scanning head is guided through an opening in a cavity,
wherein the cavity (40) is covered with an elastically extensible two-dimensionally extending, as viewed across the surface, or film-like material that is pressed against the cavity (40), and
wherein the cavity (40) is reduced in volume prior to scanning.

20. The method according to claim 19,
wherein the cavity (40) is reduced in a controlled manner regarding a volume of the cavity (40) and a shape of a reference pattern (26) of the film-like material is acquired and/or is measured.

21. The method according to claim 16,
wherein the image-recording device acquires the volume increase of the balloon and/or the extension of the balloon film (14) during inflating, and
wherein the control device (32) determines the enlargement of the reference pattern (26) during inflating before the balloon film (14) is deformed by resting against the scanning region (42) and after the balloon film is deformed by resting against the scanning region, and ally
wherein the reduction of the strength of the balloon film and/or the reference pattern (26) is calculated.

22. A control program for an image-recording device, configured to executes the method according to claim 16.

23. The image-recording device according to claim 8,
wherein the wall thickness decreases in thickness from a thickness of 200 μm to a thickness of less than 100 μm.

24. The image-recording device according to claim 23,
wherein the wall thickness decreases in thickness from a thickness of 200 μm to a thickness of 50 μm.

25. The image-recording device according to claim 12,
wherein the reference pattern (26) comprises a strength of less than 10% of the strength of the film.

26. The method according to claim 16,
wherein the cavity is a deformable cavity (40).

27. The balloon and image-recording device combination according to claim 15,
wherein the reference pattern (26) is applied to an inner surface of the balloon (12).
